(19) United States
(12) Patent Application Publication
(10) Pub. No.: US 2015/0283201 A1
(43) Pub. Date: Oct. 8, 2015

(54) IMPLANTABLE DEVICE FOR THE DELIVERY OF OCTREOTIDE AND METHODS OF USE THEREOF

(71) Applicant: Endo Pharmaceuticals Solutions Inc., Malvern, PA (US)

(72) Inventors: Petr Kuzma, Princeton, NJ (US); Harry Quandt, Bensalem, PA (US)

(73) Assignee: Endo Pharmaceuticals Solutions Inc., Malvern, PA (US)

(21) Appl. No.: 14/534,415

(22) Filed: Nov. 6, 2014

Related U.S. Application Data
(63) Continuation of application No. 13/121,152, filed on Jun. 16, 2011, now abandoned.
(60) Provisional application No. 61/101,552, filed on Sep. 30, 2008.

(51) Int. Cl. A61K 38/08; A61K 47/34; A61K 47/12; A61K 9/00

(52) U.S. Cl. CPC A61K 38/08; A61K 9/0024; A61K 9/0092; A61K 47/34; A61K 47/12

(57) ABSTRACT

This invention is related to the use of polyurethane-based polymer as a drug delivery device to deliver biologically active octreotide at a constant rate for an extended period of time and methods of manufactures thereof. The device is very bio-compatible and biostable, and is useful as an implant in patients (humans and animals) for the delivery of octreotide to tissues or organs.

### CROSS REFERENCE TO RELATED APPLICATIONS

This application claims priority to U.S. Provisional Application No. 61/101,552 filed Sep. 30, 2008, the entire disclosure of which is incorporated herein by reference.

### BACKGROUND

Due to its excellent biocompatibility, biostability and physical properties, polyurethane or polyurethane-containing polymers have been used to fabricate a large number of implantable devices, including pacemaker leads, artificial hearts, heart valves, stent coverings, artificial tendons, arteries and veins. Formulations for delivery of active agents using polyurethane implantable devices, however, require a liquid medium or carrier for the diffusion of the drug at a zero order rate.

### SUMMARY

Described herein are methods and compositions based on the unexpected discovery that solid formulations comprising one or more active agents can be used at the core of a polyurethane implantable device such that the active agent is released in a controlled-release, zero-order manner from the implantable device. The active agents and polyurethane coating can be selected based on various physical parameters, and then the release rate of the active from the implantable device can be optimized to a clinically-relevant release rate based on clinical and/or in vitro trials.

One embodiment is directed to a method for delivering a formulation comprising an effective amount of octreotide to a subject, comprising: implanting an implantable device into the subject, wherein the implantable device comprises octreotide surrounded by a polyurethane based polymer. In a particular embodiment, the polyurethane based polymer is selected from the group consisting of: a Tecophilic® polymer, a Tecoflex® polymer and a Carbothane® polymer. In a particular embodiment, the polyurethane based polymer is a Tecophilic® polymer with an equilibrium water content of at least about 24%. In a particular embodiment, the polyurethane based polymer is a Tecoflex® polymer with a flex modulus of about 2,300.

One embodiment is directed to a drug delivery device for the controlled release of octreotide over an extended period of time to produce local or systemic pharmacological effects, comprising: a) a polyurethane based polymer formed to define a hollow space; and b) a solid drug formulation comprising a formulation comprising octreotide and optionally one or more pharmaceutically acceptable carriers, wherein the solid drug formulation is in the hollow space, and wherein the device provides a desired release rate of octreotide from the device after implantation. In a particular embodiment, the drug delivery device is conditioned and primed under conditions chosen to match the water solubility characteristics of the at least one active agent. In a particular embodiment, the pharmaceutically acceptable carrier is stearic acid. In a particular embodiment, the polyurethane based polymer is selected from the group consisting of: a Tecophilic® polymer, a Tecoflex® polymer and a Carbothane® polymer. In a particular embodiment, the polyurethane based polymer is a Tecophilic® polymer with an equilibrium water content of at least about 24%. In a particular embodiment, the polyurethane based polymer is a Tecoflex® polymer with a flex modulus of about 2,300. In a particular embodiment, the appropriate conditioning and priming parameters can be selected to establish the desired delivery rates of the at least one active agent, wherein the priming parameters are time, temperature, conditioning medium and priming medium.

### BRIEF DESCRIPTION OF THE DRAWINGS

FIG. 1 is a side view of an implant with two open ends.

FIG. 2 is a side view of pre-fabricated end plugs used to plug the implants.

FIG. 3 is a side view of an implant with one open end.

### DETAILED DESCRIPTION

To take the advantage of the excellent properties of polyurethane-based polymers, the present invention is directed to the use of polyurethane-based polymers as drug delivery devices for releasing drugs at controlled rates for an extended period of time to produce local or systemic pharmacological effects. The drug delivery device can comprise a cylindrically-shaped reservoir surrounded by polyurethane-based polymer that controls the delivery rate of the drug inside the reservoir. The reservoir contains a formulation, e.g., a solid formulation, comprising one or more active ingredients and, optionally, pharmaceutically acceptable carriers. The carriers are formulated to facilitate the diffusion of the active ingredients through the polymer and to ensure the stability of the drugs inside the reservoir.

A polyurethane is any polymer consisting of a chain of organic units joined by urethane links. Polyurethane polymers are formed by reacting a monomer containing at least two isocyanate functional groups with another monomer containing at least two alcohol groups in the presence of a catalyst. Polyurethane formulations cover an extremely wide range of stiffness, hardness, and densities.

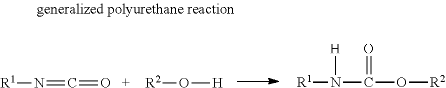

Polyurethanes are in the class of compounds called “reaction polymers,” which include epoxies, unsaturated polyesters and phenolics. A urethane linkage is produced by reacting an isocyanate group, —N═C═O with a hydroxyl (alcohol) group, —OH. Polyurethanes are produced by the polyaddition reaction of a polyisocyanate with a polyalcohol (polyol) in the presence of a catalyst and other additives. In this case, a polyisocyanate is a molecule with two or more isocyanate functional groups, R—(N═C═O)_{n≧2} and a polyol is a molecule with two or more hydroxyl functional groups, R′—(OH)_{n≧2}. The reaction product is a polymer containing the urethane linkage, —RNHCOOR′—. Isocyanates react with any molecule that contains an active hydrogen. Importantly, isocyanates react with water to form a urea linkage and carbon dioxide gas; they also react with polyetheramines to form polyureas.

Polyurethanes are produced commercially by reacting a liquid isocyanate with a liquid blend of polyols, catalyst, and other additives. These two components are referred to as a polyurethane system, or simply a system. The isocyanate is commonly referred to in North. America as the “A-side” or just the “iso,” and represents the rigid backbone (or “hard segment”) of the system. The blend of polyols and other additives is commonly referred to as the “B-side” or as the “poly,” and represents the functional section (or “soft segment”) of the system. This mixture might also be called a “resin” or “resin blend.” Resin blend additives can include chain extenders, cross linkers, surfactants, flame retardants, blowing agents, pigments and fillers. In drug delivery applications, the “soft segments” represent the section of the polymer that imparts the characteristics that determine the diffusivity of an active pharmaceutical ingredient (API) through that polymer.

The elastomeric properties of these materials are derived from the phase separation of the hard and soft copolymer segments of the polymer, such that the urethane hard segment domains serve as cross-links between the amorphous polyether (or polyester) soft segment domains. This phase separation occurs because the mainly non-polar, low-melting soft segments are incompatible with the polar, high-melting hard segments. The soft segments, which are formed from high molecular weight polyols, are mobile and are normally present in coiled formation, while the hard segments, which are formed from the isocyanate and chain extenders, are stiff and immobile. Because the hard segments are covalently coupled to the soft segments, they inhibit plastic flow of the polymer chains, thus creating elastomeric resiliency. Upon mechanical deformation, a portion of the soft segments are stressed by uncoiling, and the hard segments become aligned in the stress direction. This reorientation of the hard segments and consequent powerful hydrogen-bonding contributes to high tensile strength, elongation, and tear resistance values.

The polymerization reaction is catalyzed by tertiary amines, such as, for example, dimethylcyclohexylamine, and organometallic compounds, such as, for example, dibutyltin dilaurate or bismuth octanoate. Furthermore, catalysts can be chosen based on whether they favor the urethane (gel) reaction, such as, for example, 1,4-diazabicyclo[2.2.2]octane (also called DABCO or TEDA), or the urea (blow) reaction, such as bis-(2-dimethylaminoethyl)ether, or specifically drive the isocyanate trimerization reaction, such as potassium octoate.

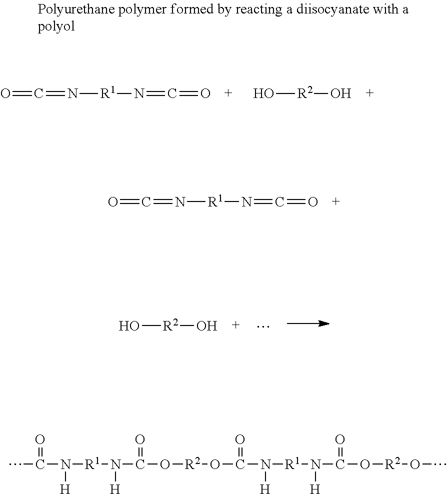

Isocyanates with two or more functional groups are required for the formation of polyurethane polymers. Volume wise, aromatic isocyanates account for the vast majority of global diisocyanate production. Aliphatic and cycloaliphatic isocyanates are also important building blocks for polyurethane materials, but in much smaller volumes. There are a number of reasons for this. First, the aromatically-linked isocyanate group is much more reactive than the aliphatic one. Second, aromatic isocyanates are more economical to use. Aliphatic isocyanates are used only if special properties are required for the final product. Light stable coatings and elastomers, for example, can only be obtained with aliphatic isocyanates. Aliphatic isocyanates also are favored in the production of polyurethane biomaterials due to their inherent stability and elastic properties.

Examples of aliphatic and cycloaliphatic isocyanates include, for example, 1,6-hexamethylene diisocyanate (HDI), 1-isocyanato-3-isocyanatomethyl-3,5,5-trimethyl-cyclohexane (isophorone diisocyanate, IPDI), and 4,4′-diisocyanato dicyclohexylmethane (H12MDI). They are used to produce light stable, non-yellowing polyurethane coatings and elastomers. H12MDI prepolymers are used to produce high performance coatings and elastomers with optical clarity and hydrolysis resistance. Tecoflex®, Tecophilic® and Carbothane® polyurethanes are all produced from H12MDI prepolymers.

Polyols are higher molecular weight materials manufactured from an initiator and monomeric building blocks, and, where incorporated into polyurethane systems, represent the “soft segments” of the polymer. They are most easily classified as polyether polyols, which are made by the reaction of epoxides (oxiranes) with an active hydrogen containing starter compounds, or polyester polyols, which are made by the polycondensation of multifunctional carboxylic acids and hydroxyl compounds.

Tecoflex® polyurethanes and Tecophilic® polyurethanes are cycloaliphatic polymers and are of the types produced from polyether-based polyols. For the Tecoflex® polyurethanes, the general structure of the polyol segment is represented as,

O—(CH₂—CH₂—CH₂—CH₂)ₓ—O—

whereby an increase in “x” represents a increase in flexibility (decreased “Flex Modulus”; “FM”), yielding FM ranging from about 1000-92,000 psi. From the standpoint of drug release from these materials, the release of a relatively hydrophobic API decreases as the FM increases.

For the Tecophilic® (hydrophilic) polyurethanes, the general structure of the polyol segment is represented as,

—[O—(CH₂)ₙ]ₓ—O—

whereby increases in “n” and “x” represent variations in hydrophilicity, and yield equilibrium water contents (% EWC) ranging from about 5%-43%. From the standpoint of drug release from these materials, the release of a relatively hydrophilic API increases as the % EWC increases.

Specialty polyols include, for example, polycarbonate polyols, polycaprolactone polyols, polybutadiene polyols, and polysulfide polyols.

Carbothane® polyurethanes are cycloaliphatic polymers and are of the types produced from polycarbonate-based polyols. The general structure of the polyol segment is represented as,

O—[(CH₂)₆—CO₃]ₙ—(CH₂)—O—

whereby an increase in “n” represents a increase in flexibility (decreased FM), yielding FM ranging from about 620-92,000 psi. From the standpoint of drug release from these materials, the release of a relatively hydrophobic API will decrease as the FM increases.

Chain extenders and cross linkers are low molecular weight hydroxyl- and amine-terminated compounds that play an important role in the polymer morphology of polyurethane fibers, elastomers, adhesives and certain integral skin and microcellular foams. Examples of chain extenders include, for example, ethylene glycol, 1,4-butanediol (1,4-BDO or BDO), 1,6-hexanediol, cyclohexane dimethanol and hydroquinone bis(2-hydroxyethyl) ether (HQEE). All of these glycols form polyurethanes that phase separate well, form well-defined hard segment domains, and are melt processable. They are all suitable for thermoplastic polyurethanes with the exception of ethylene glycol, since its derived bis-phenyl urethane undergoes unfavorable degradation at high hard segment levels. Tecophilic®, Tecoflex® and Carbothane® polyurethanes all incorporate the use of 1,4-butanediol as the chain extender.

The current invention provides a drug delivery device that can achieve the following objectives: a controlled-release rate (e.g., zero-order release rate) to maximize therapeutic effects and minimize unwanted side effects, an easy way to retrieve the device if it is necessary to end the treatment, an increase in bioavailability with less variation in absorption and no first pass metabolism.

The release rate of the drug is governed by the Fick's Law of Diffusion as applied to a cylindrically shaped reservoir device (cartridge). The following equation describes the relationship between different parameters:

$$\frac{d M}{d t} = \frac{2 π h p Δ C}{ln ( {r}_{o} / {r}_{i} )}$$

where:
dM/dt: drug release rate;
h: length of filled portion of device;
ΔC: concentration gradient across the reservoir wall;
rₒ/rᵢ: ratio of outside to inside radii of device; and
p: permeability coefficient of the polymer used.

The permeability coefficient is primarily regulated by the hydrophilicity or hydrophobicity of the polymer, the structure of the polymer, and the interaction of drug and the polymer. Once the polymer and the active ingredient are selected, p is a constant, h, ro, and rᵢ are fixed and kept constant once the cylindrically-shaped device is produced. ΔC is maintained constant.

To keep the geometry of the device as precise as possible, the device, e.g., a cylindrically-shaped device, can be manufactured through precision extrusion or precision molding process for thermoplastic polyurethane polymers, and reaction injection molding or spin casting process for thermosetting polyurethane polymers.

The cartridge can be made with either one end closed or both ends open. The open end can be plugged with, for example, pre-manufactured end plug(s) to ensure a smooth end and a solid seal, or, in the case of thermoplastic polyurethanes, by using heat-sealing techniques known to those skilled in the art. The solid actives and carriers can be compressed into pellet form to maximize the loading of the actives.

To identify the location of the implant, radiopaque material can be incorporated into the delivery device by inserting it into the reservoir or by making it into end plug to be used to seal the cartridge.

Once the cartridges are sealed on both ends with the filled reservoir, they are optionally conditioned and primed for an appropriate period of time to ensure a constant delivery rate.

The conditioning of the drug delivery devices involves the loading of the actives (drug) into the polyurethane-based polymer that surrounds the reservoir. The priming is done to stop the loading of the drug into the polyurethane-based polymer and thus prevent loss of the active before the actual use of the implant. The conditions used for the conditioning and priming step depend on the active, the temperature and the medium in which they are carried out. The conditions for the conditioning and priming may be the same in some instances.

The conditioning and priming step in the process of the preparation of the drug delivery devices is done to obtain a determined rate of release of a specific drug. The conditioning and priming step of the implant containing a hydrophilic drug can be carried out in an aqueous medium, e.g., in a saline solution. The conditioning and priming step of a drug delivery device comprising a hydrophobic drug is usually carried out in a hydrophobic medium such as, for example, an oil-based medium. The conditioning and priming steps can be carried out by controlling three specific factors, namely the temperature, the medium and the period of time.

A person skilled in the art would understand that the conditioning and priming step of the drug delivery device is affected by the medium in which the device is placed. A hydrophilic drug can be conditioned and primed, for example, in an aqueous solution, e.g., in a saline solution. Octreotide implants, for example, can be conditioned and primed in saline solution, more specifically, conditioned in saline solution of 0.9% sodium content and primed in saline solution of 1.8% sodium chloride content.

The temperature used to condition and prime the drug delivery device can vary across a wide range of temperatures, e.g., about 37° C.

The time period used for the conditioning and priming of the drug delivery devices can vary from about a single day to several weeks depending on the release rate desired for the specific implant or drug. The desired release rate is determined by one of skill in the art with respect to the particular active agent used in the pellet formulation.

A person skilled in the art will understand the steps of conditioning and priming the implants are to optimize the rate of release of the drug contained within the implant. As such, a shorter time period spent on the conditioning and the priming of a drug delivery device results in a lower rate of release of the drug compared to a similar drug delivery device that has undergone a longer conditioning and priming step.

The temperature in the conditioning and priming step will also affect the rate of release in that a lower temperature results in a lower rate of release of the drug contained in the drug delivery device when compared to a similar drug delivery device that has undergone a treatment at a higher temperature.

Similarly, in the case of aqueous solutions, e.g., saline solutions, the sodium chloride content of the solution determines what type of rate of release will be obtained for the drug delivery device. More specifically, a lower content of sodium chloride results in a higher rate of release of drug when compared to a drug delivery device that has undergone a conditioning and priming step where the sodium chloride content was higher.

The same conditions apply for hydrophobic drugs where the main difference in the conditioning and priming step is that the conditioning and priming medium is a hydrophobic medium, more specifically an oil-based medium.

Octreotide is an octapeptide that mimics natural somatostatin; although it is a more potent inhibitor of growth hormone, glucagon, and insulin than the natural hormone. Octreotide can be used to treat, for example, acromegaly, diarrhea and flushing episodes associated with carcinoid syndrome, diarrhea in patients with vasoactive intestinal peptide-secreting tumors (VIPomas), severe, refractory diarrhea from other causes, prolonged recurrent hypoglycemia after sulfonylurea overdose, infants with nesidioblastosis to help decrease insulin hypersecretion, esophageal varices, chronic pancreatitis, thymic neoplasms, hypertrophic pulmonary osteoarthropathy (HPOA), secondary to non-small cell lung carcinoma, and pain associated with HPOA. Effective levels of octreotide in the blood are known and established and can range, for example, about 0.1 to about 8 ng/ml, from about 0.25 to about 6 ng/ml or about 0.3 to about 4 ng/ml range.

The current invention focuses on the application of polyurethane-based polymers, thermoplastics or thermosets, to the creation of implantable drug devices to deliver biologically active compounds at controlled rates for prolonged period of time. Polyurethane polymers can be made into, for example, cylindrical hollow tubes with one or two open ends through extrusion, (reaction) injection molding, compression molding, or spin-casting (see e.g., U.S. Pat. Nos. 5,266,325 and 5,292,515), depending on the type of polyurethane used.

Thermoplastic polyurethane can be processed through extrusion, injection molding or compression molding. Thermoset polyurethane can be processed through reaction injection molding, compression molding, or spin-casting. The dimensions of the cylindrical hollow tube should be as precise as possible.

Polyurethane-based polymers are synthesized from multi-functional polyols, isocyanates and chain extenders. The characteristics of each polyurethane can be attributed to its structure.

Thermoplastic polyurethanes are made of macrodials, diisocyanates, and difunctional chain extenders (e.g., U.S. Pat. Nos. 4,523,005 and 5,254,662). Macrodials make up the soft domains. Diisocyanates and chain extenders make up the hard domains. The hard domains serve as physical crosslinking sites for the polymers. Varying the ratio of these two domains can alter the physical characteristics of the polyurethanes, e.g., the flex modulus.

Thermoset polyurethanes can be made of multifunctional (greater than difunctional) polyols and/or isocyanates and/or chain extenders (e.g., U.S. Pat. Nos. 4,386,039 and 4,131,604). Thermoset polyurethanes can also be made by introducing unsaturated bonds in the polymer chains and appropriate crosslinkers and/or initiators to do the chemical crosslinking (e.g., U.S. Pat. No. 4,751,133). By controlling the amounts of crosslinking sites and how they are distributed, the release rates of the actives can be controlled.

Different functional groups can be introduced into the polyurethane polymer chains through the modification of the backbones of polyols depending on the properties desired. Where the device is used for the delivery of water soluble drugs, hydrophilic pendant groups such as ionic, carboxyl, ether, and hydroxy groups are incorporated into the polyols to increase the hydrophilicity of the polymer (e.g., U.S. Pat. Nos. 4,743,673 and 5,354,835). Where the device is used for the delivery of hydrophobic drugs, hydrophobic pendant groups such as alkyl, siloxane groups are incorporated into the polyols to increase the hydrophobicity of the polymer (e.g., U.S. Pat. No. 6,313,254). The release rates of the actives can also be controlled by the hydrophilicity/hydrophobicity of the polyurethane polymers.

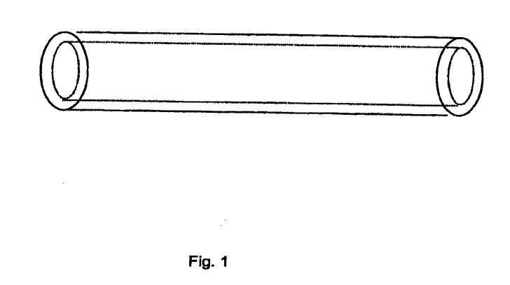

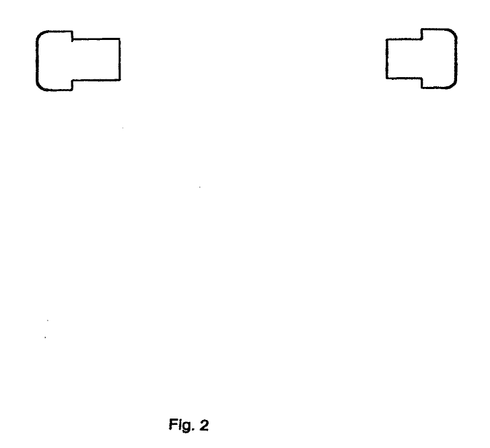

For thermoplastic polyurethanes, precision extrusion and injection molding are the preferred choices to produce two open-end hollow tubes (FIG. 1) with consistent physical dimensions. The reservoir can be loaded freely with appropriate formulations containing actives and carriers or filled with pre-fabricated pellets to maximize the loading of the actives. One open end needs to be sealed first before the loading of the formulation into the hollow tube. To seal the two open ends, two pre-fabricated end plugs (FIG. 2) can be used. The sealing step can be accomplished through the application of heat or solvent or any other means to seal the ends, preferably permanently.

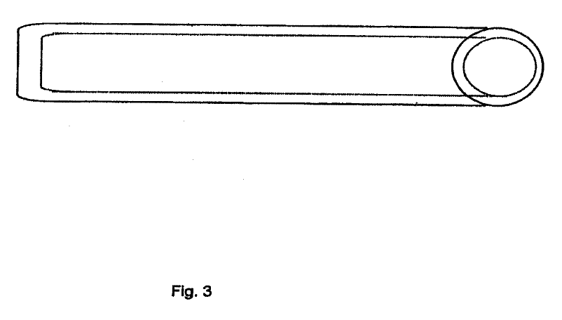

For thermoset polyurethanes, precision reaction injection molding or spin casting is the preferred choice depending on the curing mechanism. Reaction injection molding is used if the curing mechanism is carried out through heat and spin casting is used if the curing mechanism is carried out through light and/or heat. Hollow tubes with one open end (FIG. 3), for example, can be made by spin casting. Hollow tubes with two open ends, for example, can be made by reaction injection molding. The reservoir can be loaded in the same way as the thermoplastic polyurethanes.

To seal an open end, an appropriate light-initiated and/or heat-initiated thermoset polyurethane formulation can be used to fill the open end, and this is cured with light and/or heat. A pre-fabricated end plug, for example, can also be used to seal the open end by applying an appropriate light-initiated and/or heat-initiated thermoset polyurethane formulation on to the interface between the pre-fabricated end plug and the open end, and curing it with the light and/or heat or any other means to seal the ends, preferably permanently.

The final process involves the conditioning and priming of the implants to achieve the delivery rates required for the actives. Depending upon the types of active ingredient, hydrophilic or hydrophobic, the appropriate conditioning and priming media is chosen. Water-based media are preferred for hydrophilic actives, and oil-based media are preferred for hydrophobic actives.

As a person skilled in the art would readily know many changes can be made to the preferred embodiments of the invention without departing from the scope thereof. It is intended that all matter contained herein be considered illustrative of the invention and not it a limiting sense.

### EXEMPLIFICATION

### Example 1

Tecophilic® polyurethane polymer tubes are supplied by Thermedics Polymer Products and manufactured through a precision extrusion process. Tecophilic® polyurethane is a family of aliphatic polyether-based thermoplastic polyurethane that can be formulated to different equilibrium water contents (EWC) of up to 150% of the weight of dry resin. Extrusion grade formulations are designed to provide maximum physical properties of thermoformed tubing or other components. An exemplary tube and end cap structures are depicted in FIGS. 1-3.

The physical data for the polymers is provided below as made available by Thermedics Polymer Product (tests conducted as outlined by American Society for Testing and Materials (ASTM), Table 1).

TABLE 1
Tecophilic ® Typical Physical Test Data

| | | HP- | HP- | HP- | HP- |
|---|---|---|---|---|---|
| | ASTM | 60D-20 | 60D-35 | 60D-60 | 93A-100 |
| Durometer | D2240 | 43D | 42D | 41D | 83A |
| (Shore | | | | | |
| Hardness) | | | | | |
| Spec Gravity | D792 | 1.12 | 1.12 | 1.15 | 1.13 |
| Flex Modulus | D790 | 4,300 | 4,000 | 4,000 | 2,900 |
| (psi) | | | | | |
| Ultimate | D412 | 8,900 | 7,800 | 8,300 | 2,200 |
| Tensile | | | | | |
| Dry (psi) | | | | | |
| Ultimate | D412 | 5,100 | 4,900 | 3,100 | 1,400 |
| Tensile | | | | | |
| Wet (psi) | | | | | |
| Elongation | D412 | 430 | 450 | 500 | 1,040 |
| Dry (%) | | | | | |
| Elongation | D412 | 390 | 390 | 300 | 620 |
| Wet (%) | | | | | |

### Example 2

Tables 2A-D show release rates of octreotide from three different classes of polyurethane compounds (Tecophilic®, Tecoflex® and Carbothane®). The release rates have been normalized to surface area of the implant, thereby adjusting for slight differences in the size of the various implantable devices. Octreotide is water-soluble as indicated by the Log P value; for the purposes of the data provided, a Log P value of greater than about 2.0 is considered to be not readily soluble in aqueous solution. The polyurethanes were selected to have varying affinities for water soluble active agents and varying flexibility (as indicated by the variation in flex modulus).

For applications of the polyurethanes useful for the devices and methods described herein, the polyurethane exhibits physical properties suitable for the octreotide formulation to be delivered. Polyurethanes are available or can be prepared, for example, with a range of EWCs or flex moduli (Table 2). Tables 2A-B show normalized release rates for various active ingredients from polyurethane compounds. Tables 2C-D show the non-normalized release rates for the same active ingredients, together with implant composition.

TABLE 2A

| | | Polyurethane Type | | | | |
|---|---|---|---|---|---|---|
| | | Tecophilic | | | | |
| | | Polyurethane Grade | | | | |
| | | HP-60D-60 | HP-60D-35 | HP-60D-20 | HP-60D-10 | HP-60D-05 |
| | Relative Water | % EWC/Flex Modulus | | | | |
| Active | Solubility | 31% EWC | 24% EWC | 15% EWC | 8.7% EWC | 5.5% EWC |
| Octreotide | Very soluble, | — | 2022 μg/day/cm² | 758 μg/day/cm² | 11 μg/day/cm² | 0 |
| Acetate | Log P = 0.43 | | 2% SA | 5% HPC, 2% SA; | 10% HPC, 2% SA, | 10% HPC, 2% |
| (M.W. 1019) | | | 50 mg API | 50 mg API | 50 mg API | SA, 50 mg API |

TABLE 2B

| | | Polyurethane Type | | |
|---|---|---|---|---|
| | | Tecoflex | | |
| | | Polyurethane Grade | | |
| | Relative | EG-85A | EG 100A | EG-65D |
| | Water | % EWC/Flex Modulus | | |
| Active | Solubility | F.M.: 2,300 | F.M.: 10,000 | F.M.: 37,000 |
| Octreotide | Very | 16 μg/day/cm² | — | — |
| Acetate | soluble, | 10% HPC, | | |
| (M.W. 1019) | Log P = 0.43 | 2% SA, | | |
| | | 50 mg API | | |

TABLE 2C

| | | Polyurethane | | | | |
|---|---|---|---|---|---|---|
| | | Tecophilic | | | | |
| | | Grade | | | | |
| | | HP-60D-60 | HP-60D-35 | HP-60D-20 | HP-60D-10 | HP-60D-05 |
| | Relative Water | % EWC | | | | |
| Active | Solubility | 31% EWC | 24% EWC | 15% EWC | 8.7% EWC | 5.5% EWC |
| Octreotide | Very soluble, | — | 400 μg/day | 1500 μg/day | 25 μg/day | 0 |
| Acetate | Log P = 0.43 | | ID: 1.80 mm | ID: 1.80 mm | ID: 1.83 mm | |
| (M.W. 1019) | | | Wall: 0.30 mm | Wall: 0.30 mm | Wall: 0.30 mm | |
| | | | L: 30 mm | L: 30 mm | L: 34 mm | |
| | | | 1.978 cm² | 1.978 cm² | 2.274 cm² | |

TABLE 2D

| | | Polyurethane Type | | |
|---|---|---|---|---|
| | | Tecoflex | | |
| | | Polyurethane Grade | | |
| | Relative | EG-85A | EG 100A | EG-65D |
| | Water | Flex Modulus | | |
| Active | Solubility | F.M.: 2,300 | F.M.: 10,000 | F.M.: 37,000 |
| Octreotide | Very | 30 μg/day | — | — |
| Acetate | soluble, | ID: 1.85 mm | | |
| (M.W. 1019) | Log P = 0.43 | Wall: 0.20 mm | | |
| | | L: 30 mm | | |
| | | 1.931 cm² | | |

The solubility of an active agent in an aqueous environment can be measured and predicted based on its partition coefficient (defined as the ratio of concentration of compound in aqueous phase to the concentration in an immiscible solvent). The partition coefficient (P) is a measure of how well a substance partitions between a lipid (oil) and water. The measure of solubility based on P is often given as Log P. In general, solubility is determined by Log P and melting point (which is affected by the size and structure of the compounds). Typically, the lower the Log P value, the more soluble the compound is in water. It is possible, however, to have compounds with high Log P values that are still soluble on account of, for example, their low melting point. It is similarly possible to have a low Log P compound with a high melting point, which is very insoluble.

The flex modulus for a given polyurethane is the ratio of stress to strain. It is a measure of the “stiffness” of a compound. This stiffness is typically expressed in Pascals (Pa) or as pounds per square inch (psi).

The elution rate of an active agent from a polyurethane compound can vary on a variety of factors including, for example, the relative hydrophobicity/hydrophilicity of the polyurethane (as indicated, for example, by log P), the relative “stiffness” of the polyurethane (as indicated, for example, by the flex modulus), and/or the molecular weight of the active agent to be released.

### EQUIVALENTS

The present disclosure is not to be limited in teems of the particular embodiments described in this application, which are intended as illustrations of various aspects. Many modifications and variations can be made without departing from the spirit and scope of the disclosure, as will be apparent to those skilled in the art. Functionally equivalent methods, systems, and apparatus within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting. As will be understood by one skilled in the art, for any and all purposes, such as in terms of providing a written description, all ranges disclosed herein also encompass any and all possible subranges and combinations of subranges thereof.

While various aspects and embodiments have been disclosed herein, other aspects and embodiments will be apparent to those skilled in the art. All references cited herein are incorporated by reference in their entireties.

What is claimed is:

1. A method for delivering a formulation comprising an effective amount of octreotide to a subject, comprising: implanting an implantable device into the subject, wherein the implantable device comprises octreotide surrounded by a polyurethane-based polymer.

2. The method of claim 1, wherein the polyurethane-based polymer is selected from the group consisting of: a Tecophilic® polymer, a Tecoflex® polymer and a Carbothane® polymer.

3. The method of claim 2, wherein the polyurethane-based polymer is a Tecophilic® polymer with an equilibrium water content of at least about 24%.

4. The method of claim 2, wherein the polyurethane-based polymer is a Tecoflex® polymer with a flex modulus of about 2,300.

5. A drug delivery device for the controlled release of octreotide over an extended period of time to produce local or systemic pharmacological effects, comprising:
a) a polyurethane-based polymer formed to define a hollow space; and
b) a solid drug formulation comprising octreotide and optionally one or more pharmaceutically-acceptable carriers,
wherein the solid drug formulation is in the hollow space, and wherein the device provides a desired release rate of octreotide from the device after implantation.

6. The drug delivery device of claim 5, wherein the drug delivery device is conditioned and primed under conditions chosen to match the water solubility characteristics of the at least one active agent.

7. The drug delivery device of claim 6, wherein the pharmaceutically-acceptable carrier is stearic acid.

8. The drug delivery device of claim 7, wherein the polyurethane-based polymer is selected from the group consisting of: a Tecophilic® polymer, a Tecoflex® polymer and a Carbothane® polymer.

9. The drug delivery device of claim 8, wherein the polyurethane-based polymer is a Tecophilic® polymer with an equilibrium water content of at least about 24%.

10. The drug delivery device of claim 8, wherein the polyurethane-based polymer is a Tecoflex® polymer with a flex modulus of about 2,300.

11. The drug delivery device of claim 5, wherein the appropriate conditioning and priming parameters can be selected to establish the desired delivery rates of the at least one active agent, wherein the priming parameters are time, temperature, conditioning medium and priming medium.
